(19) United States
(12) Patent Application Publication
(10) Pub. No.: US 2023/0311142 A1
(43) Pub. Date: Oct. 5, 2023

(54) Atomizing Container and Atomizing Apparatus

(71) Applicants: Guangzhou Chiyang Scent Technology Co., LTD., Guangzhou (CN); Arizona Air-Scent, Inc., Scottsdale, AZ (US)

(72) Inventors: Xiaoyang Gao; Sam Habbel, Scottsdale, AZ (US)

(21) Appl. No.: 18/247,327

(22) PCT Filed: Aug. 10, 2022
(86) PCT No.: PCT/CN2022/111495

§ 371 Date: Mar. 30, 2023

(30) Foreign Application Priority Data
Aug. 11, 2021 (CN) 202121875154.7
Aug. 11, 2021 (CN) 202121875207.5

(51) Int. Cl. B05B 7/24

(52) U.S. Cl. CPC B05B 7/2489

(57) ABSTRACT

An atomization container includes a container body and an atomizer, wherein the container body has an accommodating cavity therein, the atomization container also has a gas inlet passage and a mist outlet passage, the gas inlet passage has one end configured to be connected with an external gas source and the other end connected with the atomizer for providing gas flow for the atomizer, the atomizer is configured to mix and atomize the gas flow provided through the gas inlet passage and liquid in the accommodating cavity to form mist, the mist outlet passage is configured to discharge the mist out of the container body, and the atomizer is located in the accommodating cavity.

The present application claims priority to Chinese Patent Application Nos. 202121875207.5 and 202121875154.7, filed with China National Intellectual Property Administration on Aug. 11, 2021, the entire content of which is incorporated herein by reference.

### TECHNICAL FIELD

The present application relates to the field of atomization devices, and particularly to an atomization container and an atomization apparatus which are capable of atomizing liquid.

### BACKGROUND ART

With a continuous improvement of living standards, more and more people pay more attention to quality of life, and people need both beautiful and clean living and working places and clean air without odor pollution. In order to improve an air environment, people mostly adopt methods of spraying a perfume or essential oil in a room, or the like, to improve air quality in a space, thereby keeping air in the room fresh, eliminating a peculiar smell, and achieving sterilization and disinfection effects. However, the spray of the perfume or essential oil in the room is troublesome and required to be performed frequently to maintain a fragrance, and since sprayed perfume particles are large and have a small drifting range, the method is inefficient and wasteful. Based on this, an atomizer capable of automatically spraying is invented.

The existing atomizer generally includes an essential oil bottle and an atomizing head, and the essential oil bottle is connected with the atomizing head. In use, the atomizing head is required to be connected with the essential oil bottle, such that the atomizer can atomize essential oil in the essential oil bottle; then, the atomizing head and the essential oil bottle are mounted in an atomizer housing, and therefore, the use is inconvenient.

### SUMMARY

Based on this, it is necessary to provide an atomization container and an atomization apparatus which are convenient to use and have a good atomization effect.

In an aspect, the present application provides an atomization container, including: a container body and an atomizer, wherein the container body has an accommodating cavity therein, the atomization container also has a gas inlet passage and a mist outlet passage, the gas inlet passage has one end configured to be connected with an external gas source and the other end connected with the atomizer for providing gas flow for the atomizer, the atomizer is configured to mix and atomize the gas flow provided through the gas inlet passage and liquid in the accommodating cavity to form mist, the mist outlet passage is configured to discharge the mist out of the container body, and the atomizer is located in the accommodating cavity; in a first state, the gas inlet passage and the mist outlet passage are opened, such that gas flow of a gas pump may enter the atomizer through the gas inlet passage, and the mist formed by the atomizer may be discharged out of the container body through the mist outlet passage; in a second state, the gas inlet passage and/or the mist outlet passage are/is closed.

Preferably, the atomization container further includes a first cover, the first cover is connected with the container body and closes an opening of the container body, the atomizer is connected with the first cover, the gas inlet passage and the mist outlet passage are provided in the first cover, the first cover is also provided with a first valve and a second valve, the first valve is fitted with the gas inlet passage and configured to open or close the gas inlet passage, and the second valve is fitted with the mist outlet passage and configured to open or close the mist outlet passage; in a free state, the first valve and the second valve are closed, the first cover seals the container body, the second valve is movably mounted in the first cover, and the atomizer is fixed on the second valve and may move with the second valve relative to the container body.

Preferably, the first valve includes a first sealing part and a first elastic part, the first elastic part is connected with the first sealing part, the second valve includes a second sealing part and a second elastic part, the second elastic part is connected with the second sealing part, and when the first elastic part and the second elastic part are in a free state, the first sealing part closes the gas inlet passage, and the second sealing part closes the mist outlet passage; when the first sealing part and the second sealing part are pressed down by external force, the gas inlet passage communicates the atomizer with a space outside the container body, and the mist outlet passage communicates the accommodating cavity with the space outside the container body.

Preferably, a hollow mounting cylinder is provided in the first cover, openings are formed in an upper end and a lower end of the mounting cylinder, the second valve is movably mounted in the mounting cylinder, the second valve includes a vent pipe and a seal fixed on a peripheral side of the vent pipe, the gas inlet passage is formed in the vent pipe, the first valve is provided in the gas inlet passage, the mist outlet passage is formed in the mounting cylinder, and the mist outlet passage is located outside the vent pipe; an upper end of the vent pipe penetrates through the opening at the upper end of the mounting cylinder to protrude outwards, the seal is tightly fitted with an inner wall of the mounting cylinder, the atomizer is fixed at a lower end of the vent pipe, and in a first state, the seal opens the mist outlet passage, and the mist formed by the atomizer may be discharged out of the atomization container through the mist outlet passage; in a second state, the seal blocks the mist outlet passage, and the mist formed by the atomizer cannot be discharged out of the atomization container through the mist outlet passage.

Preferably, an upper segment of the vent pipe is in clearance fit with the opening at the upper end of the mounting cylinder, a connecting through hole is formed in a side wall of the mounting cylinder, the connecting through hole is communicated with the accommodating cavity, the second valve may move relative to the connecting through hole under the action of external force, and in a first state, the seal opens a passage from the connecting through hole to the opening at the upper end of the mounting cylinder, and the mist formed by the atomizer enters the mounting cylinder from the accommodating cavity through the connecting through hole, and is discharged out of the first cover through the opening at the upper end of the mounting cylinder; in a second state, the seal closes the passage from the connecting through hole to the opening at the upper end of the mounting cylinder, and the mist formed by the atomizer is sealed in the accommodating cavity.

Preferably, the atomization container further includes a third cover, the third cover is a sealing cover, the third cover is detachably connected with the first cover, and when the third cover is hermetically connected with the first cover, the atomizer, the gas inlet passage and the mist outlet passage are sealed in the accommodating cavity.

Preferably, the atomization container further includes a second cover, the second cover is provided with a top column, an auxiliary gas inlet passage, an auxiliary mist outlet passage and a mist outlet, the auxiliary gas inlet passage is provided in the top column, the mist outlet is communicated with the auxiliary mist outlet passage, and when the second cover is connected with the first cover, the top column presses down the first valve and the second valve, such that the auxiliary gas inlet passage is communicated with the gas inlet passage, the auxiliary mist outlet passage is communicated with the mist outlet passage, and the gas inlet passage and the auxiliary gas inlet passage are isolated from the mist outlet passage and the auxiliary mist outlet passage.

Preferably, a hollow mounting cylinder is provided in the first cover, openings are formed in an upper end and a lower end of the mounting cylinder, the second valve is vertically movably mounted in the mounting cylinder, the second valve includes a vent pipe and a seal fixed on a peripheral side of the vent pipe, the gas inlet passage is formed in the vent pipe, the first valve is provided in the gas inlet passage, the mist outlet passage is formed in the mounting cylinder, and the mist outlet passage is located outside the vent pipe; an upper end of the vent pipe penetrates through the opening at the upper end of the mounting cylinder to protrude outwards, the seal is tightly fitted with an inner wall of the mounting cylinder, the atomizer is fixed at a lower end of the vent pipe, and when the second cover is connected with the first cover, the top column presses down the second valve to open the mist outlet passage, and a lower end portion of the top column is inserted into the second valve and abuts against the first valve, such that the auxiliary gas inlet passage and the gas inlet passage form a butt joint and are communicated.

Preferably, the first valve includes a first sealing part and a first elastic part, the first elastic part is connected with the first sealing part, the first sealing part is a spherical sealing part, and when the lower end portion of the top column is inserted into the second valve, the first sealing part is pressed downwards, a notch is formed at a position of the lower end portion of the top column in contact with the first sealing part, and the gas inlet passage is communicated with the auxiliary gas inlet passage through the notch.

Preferably, the second cover is provided with a gas inlet connector, the gas inlet connector is connected with the auxiliary gas inlet passage, the second cover is provided with an auxiliary air outlet surrounding the mist outlet and an auxiliary air inlet located on one side of the second cover, and the auxiliary air inlet is communicated with the auxiliary air outlet.

Preferably, the atomizer has a gas passage and a liquid passage, the gas passage has a gas inlet and a gas outlet which are communicated with each other, the liquid passage has a liquid inlet and a liquid outlet which are communicated with each other, the gas outlet is adjacent to the liquid outlet, the atomizer further includes a pipette, the pipette has one end extending to a bottom of the accommodating cavity and the other end communicated with the liquid inlet, the gas inlet is connected with the gas inlet passage, the gas outlet is located above a liquid level of the accommodating cavity, and the gas flow provided by the gas inlet passage and the liquid in the accommodating cavity are mixed and atomized near the gas outlet and discharged into the accommodating cavity.

An embodiment of the present application further provides an atomization apparatus, including: an atomization apparatus body and an atomization container, wherein the atomization apparatus body is provided with a mounting hole, the atomization container is inserted into the mounting hole, and a gas pump is also provided in the atomization apparatus body; the atomization container is the atomization container according to the above embodiment, and the gas inlet passage has one end connected with the gas pump and the other end connected with the atomizer, such that the gas pump provides gas flow for the atomizer through the gas inlet passage, and the mist is discharged out of the container body through the mist outlet passage and finally discharged out of the atomization apparatus body.

Preferably, the atomizer has a gas passage and a liquid passage, the gas passage has a gas inlet and a gas outlet which are communicated with each other, the liquid passage has a liquid inlet and a liquid outlet which are communicated with each other, the gas outlet is adjacent to the liquid outlet, the atomizer further includes a pipette, the pipette has one end extending to a bottom of the accommodating cavity and the other end communicated with the liquid inlet, the gas inlet is connected with the gas inlet passage, the gas outlet is located above a liquid level of the accommodating cavity, and the gas flow provided by the gas inlet passage and the liquid in the accommodating cavity are mixed and atomized near the gas outlet and discharged into the accommodating cavity; and

the atomizer includes an atomizer body and a blocking part, and a liquid passage and a gas passage are provided on the atomizer body; a liquid outlet is provided at a tail end of the liquid passage, a gas outlet is provided at a tail end of the gas passage, and the liquid outlet is distributed on a periphery of the gas outlet; the blocking part is provided opposite to the gas outlet and the liquid outlet and spaced apart from the gas outlet and the liquid outlet by a preset distance, the blocking part refracts the gas flow emitted from the gas outlet, and the liquid outlet is located downstream of the gas outlet in the refracting direction; the atomization apparatus body includes a first body and a second body, the first body and the second body are detachably connected, and the first body is sleeved by the second body; the gas passage is formed in the first body, and the liquid passage is formed in the second body or an outer wall of the first body.

Preferably, a gas outlet connector connected with the gas pump is provided on a side wall of the mounting hole, the atomization container is provided with a gas inlet connector connected with the gas inlet passage, and when the atomization container is inserted into the mounting hole, the gas inlet connector is communicated with the gas outlet connector.

Preferably, the atomization container further includes a first cover, the first cover is connected with the container body and closes an opening of the container body, the atomizer is connected with the first cover, the gas inlet passage and the mist outlet passage are provided in the first cover, the first cover is also provided with a first valve and a second valve, the first valve is fitted with the gas inlet passage and configured to open or close the gas inlet passage, and the second valve is fitted with the mist outlet passage and configured to open or close the mist outlet passage; in a free state, the first valve and the second valve are closed, and the first cover seals the container body;
the atomization container further includes a second cover, the second cover is provided with a top column, an auxiliary gas inlet passage, an auxiliary mist outlet passage and a mist outlet, the auxiliary gas inlet passage is provided in the top column, the mist outlet is communicated with the auxiliary mist outlet passage, and when the second cover is connected with the first cover, the top column presses down the first valve and the second valve, such that the auxiliary gas inlet passage is communicated with the gas inlet passage, the auxiliary mist outlet passage is communicated with the mist outlet passage, and the gas inlet passage and the auxiliary gas inlet passage are isolated from the mist outlet passage and the auxiliary mist outlet passage; and
the gas inlet connector is provided on the second cover, the gas inlet connector is connected with the auxiliary gas inlet passage, the second cover is provided with an auxiliary air outlet surrounding the mist outlet and an auxiliary air inlet located on one side of the second cover, the auxiliary air inlet is communicated with the auxiliary air outlet, the atomization apparatus body is further provided with an auxiliary air blowing apparatus, and the auxiliary air blowing apparatus is opposite to the auxiliary air inlet and blows air into the auxiliary air inlet and out of the atomization apparatus body through the auxiliary air outlet.

Preferably, the atomization apparatus body includes an inner cylinder and a rebounder, the inner cylinder is inserted into the mounting hole, the inner cylinder is provided with a rebounding portion protruding towards a periphery of the inner cylinder, the rebounder is mounted in the atomization apparatus body, is located outside the inner cylinder, and abuts against the rebounding portion from bottom to top, the atomization container is partially inserted into the inner cylinder, the rebounder has a compressed state and a bounced state under the action of external force, and a height of the atomization container relative to the mounting hole in the bounced state of the rebounder is higher than a height of the atomization container relative to the mounting hole in the compressed state of the rebounder.

Preferably, the atomization container further includes a first cover, the first cover is connected with the container body and closes an opening of the container body, the atomizer is connected with the first cover, the gas inlet passage and the mist outlet passage are provided in the first cover, the first cover is also provided with a first valve and a second valve, the first valve is fitted with the gas inlet passage and configured to open or close the gas inlet passage, and the second valve is fitted with the mist outlet passage and configured to open or close the mist outlet passage;
the atomization container further includes a second cover, the second cover has a greater outer diameter than the first cover, the second cover is provided with a top column, an auxiliary gas inlet passage, an auxiliary mist outlet passage and a mist outlet, the auxiliary gas inlet passage is provided in the top column, the mist outlet is communicated with the auxiliary mist outlet passage, the second cover is connected with the first cover, and the top column presses down the first valve and the second valve, such that the auxiliary gas inlet passage is communicated with the gas inlet passage, the auxiliary mist outlet passage is communicated with the mist outlet passage, and the gas inlet passage and the auxiliary gas inlet passage are isolated from the mist outlet passage and the auxiliary mist outlet passage; and
the first cover is inserted into the inner cylinder, the second cover is located outside the inner cylinder, a supporting step is provided on an inner wall of the inner cylinder, and the second cover is supported on the supporting step.

Preferably, the atomization container further includes a first cover, the first cover is connected with the container body and closes an opening of the container body, the atomizer is connected with the first cover, the gas inlet passage and the mist outlet passage are provided in the first cover, the first cover is also provided with a first valve and a second valve, the first valve is fitted with the gas inlet passage and configured to open or close the gas inlet passage, and the second valve is fitted with the mist outlet passage and configured to open or close the mist outlet passage;
the atomization container further includes a second cover, the second cover is provided with a top column, an auxiliary gas inlet passage, an auxiliary mist outlet passage and a mist outlet, the auxiliary gas inlet passage is provided in the top column, the mist outlet is communicated with the auxiliary mist outlet passage, the second cover is connected with the first cover, and the top column presses down the first valve and the second valve, such that the auxiliary gas inlet passage is communicated with the gas inlet passage, the auxiliary mist outlet passage is communicated with the mist outlet passage, and the gas inlet passage and the auxiliary gas inlet passage are isolated from the mist outlet passage and the auxiliary mist outlet passage; and
the second cover is columnar, an outer side surface of the second cover fitted with the mounting hole includes a first arc surface region and a first plane region connected with the first arc surface region, an inner wall of the mounting hole is correspondingly provided with a second arc surface region and a second plane region connected with the second arc surface region, the first arc surface region and the first plane region are in correspondingly fitting contact with the second arc surface region and the second plane region respectively, and the gas inlet connector and the gas outlet connector are provided on the first plane region and the second plane region respectively.

Preferably, the atomization apparatus includes a plurality of atomization containers, a plurality of mounting holes are correspondingly formed in the atomization apparatus body, and each atomization container is connected with a corresponding gas pump, or the plurality of atomization containers are connected with the same gas pump; the atomization apparatus further includes auxiliary air blowing apparatuses, each atomization container is provided with a mist outlet and an auxiliary air outlet surrounding the mist outlet, the mist outlet is communicated with the mist outlet passage, the auxiliary air outlet of each atomization container is connected with one corresponding auxiliary air blowing apparatus, or the auxiliary air outlets of the plural atomization containers are connected with the same auxiliary air blowing apparatus.

The atomization container according to the present application is internally provided with the atomizer, and therefore more easily mounted in the atomization apparatus; moreover, the atomizer is replaced while the atomization container is replaced, such that the atomizer of the atomization apparatus is always in a good working state, and an atomization effect is better.

### BRIEF DESCRIPTION OF DRAWINGS

The above and other objects, features and advantages of the present application will become more apparent from a more detailed description of the preferred embodiments of the present application shown in the accompanying drawings. The same reference numerals refer to the same parts throughout the drawings, and the drawings are not intentionally drawn to scale, the emphasis being placed on illustrating the subject matter of the present disclosure.

FIG. 1 is a sectional view of an atomization container according to an embodiment of the present application;

FIG. 2 is a sectional view of the atomization container according to the embodiment of the present application in a use state;

FIG. 3 is a sectional view of a second cover in the embodiment of the present application;

FIG. 4 is a sectional view of a first cover in the embodiment of the present application;

FIG. 5 is an overall structural diagram of an atomizer in the embodiment of the present application;

FIG. 6 is an exploded view of the atomizer in the embodiment of the present application;

FIG. 7 is an overall structural diagram of a second body in the embodiment of the present application;

FIG. 8 is an overall structural diagram of a first body in the embodiment of the present application;

FIG. 9 is a sectional view of the atomizer in the embodiment of the present application;

FIG. 10 is a schematic structural diagram of an atomization apparatus according to an embodiment of the present application;

FIG. 11 is a schematic structural diagram of an atomization container according to another embodiment of the present application;

FIG. 12 is a sectional view of the atomization container of FIG. 11;

FIG. 13 is another partial sectional view of the atomization container of FIG. 11;

FIG. 14 is a sectional view of the atomization apparatus of FIG. 10;

FIG. 15 is a partial enlarged view of the atomization apparatus of FIG. 14;

FIG. 16 is another sectional view of the atomization apparatus of FIG. 10; and

FIG. 17 is a partial enlarged view of the atomization apparatus of FIG. 16.

### DETAILED DESCRIPTION OF EMBODIMENTS

The technical solutions of the present application will be further described in detail below in conjunction with the accompanying drawings and specific embodiments, such that those skilled in the art can better understand the present application and implement it, but the embodiments are not intended to limit the present application.

The present application provides an atomization apparatus, including: an atomization apparatus body and an atomization container, wherein the atomization apparatus body is provided with a mounting hole, the atomization container is inserted into the mounting hole, a gas pump is also provided in the atomization apparatus body, the atomization container includes a container body and an atomizer, the atomization container also has a gas inlet passage and a mist outlet passage, and the gas inlet passage has one end connected with the gas pump and the other end connected with the atomizer, such that the gas pump provides gas flow for the atomizer through the gas inlet passage; the atomizer is configured to mix and atomize the gas flow provided through the gas inlet passage and liquid in an accommodating cavity to form mist, the mist outlet passage is configured to discharge the mist out of the container body and finally out of the atomization apparatus body, a side wall of the mounting hole is provided with a gas outlet connector connected with the gas pump, the atomization container is provided with a gas inlet connector connected with the gas inlet passage, and when the atomization container is inserted into the mounting hole, the gas inlet connector is communicated with the gas outlet connector.

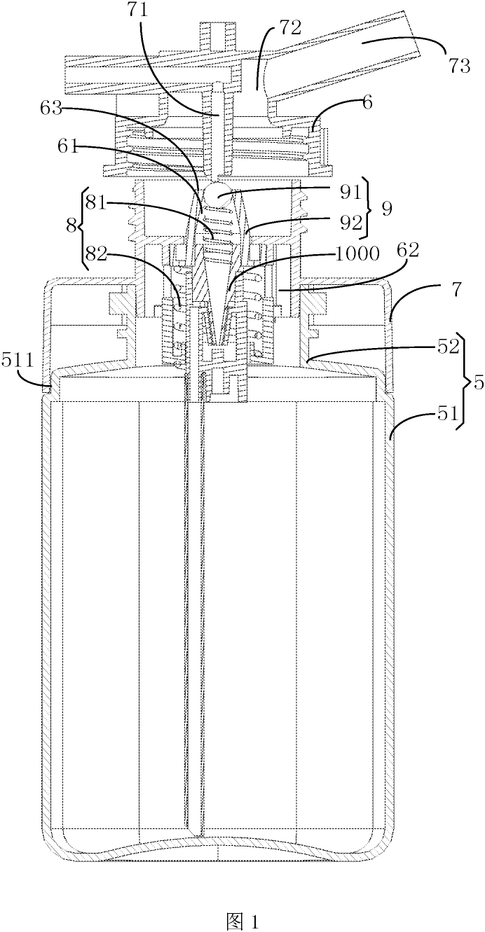

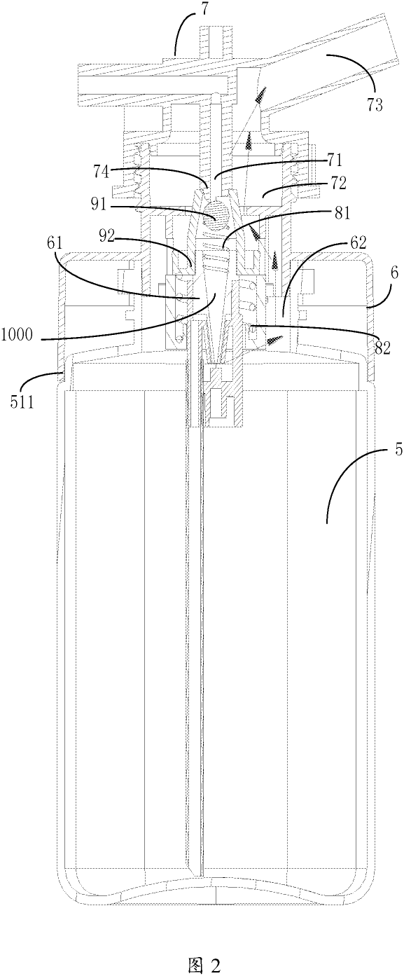

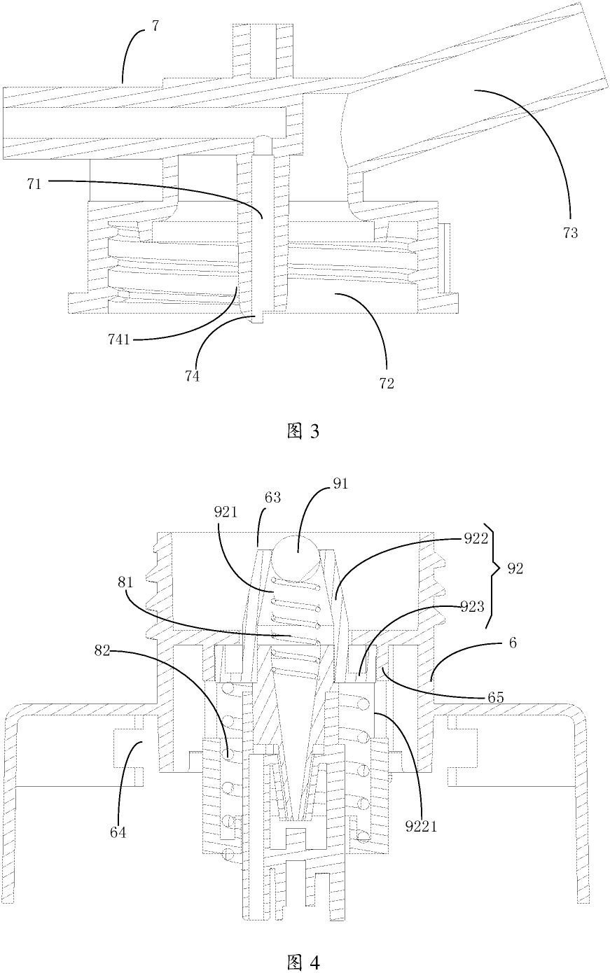

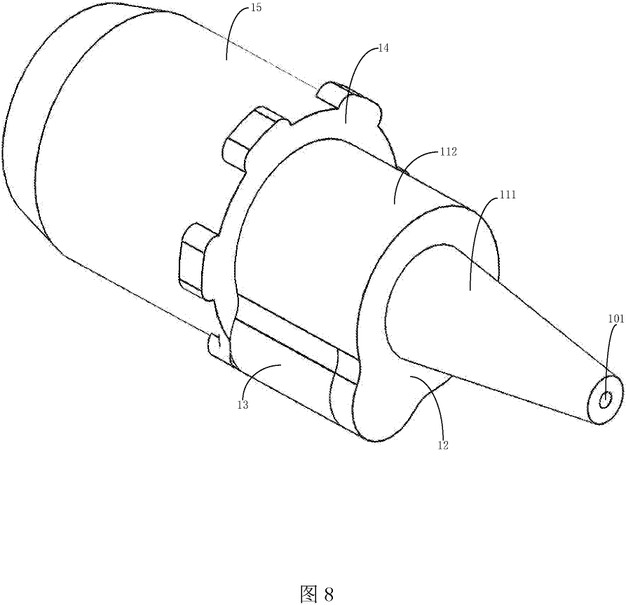

In some embodiments, as shown in FIGS. 1 to 9, the atomization container includes a container body 5 and an atomizer 1000, an accommodating cavity is formed in the container body 5, the atomizer 1000 is located in the accommodating cavity, the atomizer may be partially or completely located in the accommodating cavity, and both cases fall within the protection scope of the present application. The atomization container 5 also has a gas inlet passage 61 and a mist outlet passage 62, and the gas inlet passage 61 is configured to connect an external gas source with the atomizer 1000 to supply gas flow to the atomizer 1000. The atomizer 1000 is configured to mix and atomize the gas flow provided by the gas inlet passage 61 and liquid in the accommodating cavity to form mist. The mist outlet passage 62 is connected with the accommodating cavity and configured to discharge the mist out of the container body 5. In a first state, the gas inlet passage 61 and the mist outlet passage 62 are opened, such that the gas flow of the external gas source can enter the atomizer through the gas inlet passage, and the mist formed by the atomizer can be discharged out of a space outside the container through the mist outlet passage, generally into outside air; at this point, the atomization container is generally in a use state, and the liquid is atomized by high pressure gas introduced from the gas inlet passage 61, and the formed mist is diffused from the mist outlet passage 62. In a second state, the gas inlet passage 61 and/or the mist outlet passage 62 are/is closed, and at this point, the atomization container is generally in a non-use state to prevent the liquid in the container body 5 from flowing out.

As a further improvement to the above embodiment, the atomization container further includes a first cover 6, the first cover 6 is connected with the container body 5 and closes an opening of the container body 5, the atomizer 1000 is connected with the first cover 6, and the gas inlet passage 61 and the mist outlet passage 62 are provided on the first cover 6. Certainly, in other embodiments, the gas inlet passage 61 and the mist outlet passage 62 may be provided on the atomizer 1000 or the container body 5 or other components.

As a further improvement to the above embodiment, the first cover 6 is further provided with a first valve and a second valve, the first valve is fitted with the gas inlet passage 61 and configured to open or close the gas inlet passage 61, and the second valve is fitted with the mist outlet passage 62 and configured to open or close the mist outlet passage 62. In a free state, the first valve and the second valve are closed, and at this point, the atomization container is in the non-use state, and the liquid may be prevented from being leaked from the container body 5.

As a further improvement to the above embodiment, the first cover 6 is connected to the opening of the container body 5, and a second cover 7 is movably connected onto the first cover 6. The gas inlet passage 61 and the mist outlet passage 62 are provided in the first cover 6; the second cover 7 is provided with a top column 74, an auxiliary gas inlet passage 71, an auxiliary mist outlet passage 72 and a mist outlet 73, an elastic part 8 and a sealing part 9 are further provided in the first cover 6, and the elastic part 8 is connected with the sealing part 9; specifically, the elastic part 8 is provided at a lower portion of the sealing part, and the second cover 7 can contract the elastic part by pressing the sealing part 9. In the first state, the gas inlet passage 61 and the auxiliary gas inlet passage 71 are separated by the sealing part, the mist outlet passage 62 and the auxiliary mist outlet passage 72 are also separated by the sealing part 9, and at this point, the bottle is in a non-use state. In the second state, the top column 74 applies pressure to the sealing part, the gas inlet passage 61 and the auxiliary gas inlet passage 71 are communicated, the mist outlet passage 62 and the auxiliary mist outlet passage 72 are also communicated, and at this point, the bottle is in a use state.

Referring to FIGS. 1 to 4, as a further improvement to the above embodiment, the elastic part 8 includes a first spring 81 and a second spring 82, and the sealing part 9 includes a first sealing part 91 and a second sealing part 92. The first valve includes the first sealing part and a first elastic part, the second valve includes the second sealing part and a second elastic part, the first spring 81 is connected with the first sealing part 91, the second spring 82 is connected with the second sealing part 92, and the auxiliary gas inlet passage 71 penetrates through the top column 74. When the second cover 7 is moved towards the first cover 6, the first sealing part 91 and the second sealing part 92 are pressed by the top column 74, such that the gas inlet passage 61 is communicated with the auxiliary gas inlet passage 71, and the mist outlet passage 62 is communicated with the auxiliary mist outlet passage 72. When the second cover 7 is moved away from the first cover 6 (for example, the second cover 7 is unscrewed), the first spring 81 and the second spring 82 recover from deformation, and the first sealing part 91 and the second sealing part 92 can achieve sealing and separating effects again, so as to separate the gas inlet passage 61 from the auxiliary gas inlet passage 71 and also separate the mist outlet passage 62 from the auxiliary mist outlet passage 72. When the first elastic part 81 and the second elastic part 82 are in the free state, the first sealing part 91 closes the gas inlet passage 61, the second sealing part 92 closes the mist outlet passage 62, and when the first sealing part 91 and the second sealing part 92 are pressed down by external force, the gas inlet passage 61 communicates the atomizer 1000 with the space outside the container, and the mist outlet passage 62 communicates the accommodating cavity with the space outside the container.

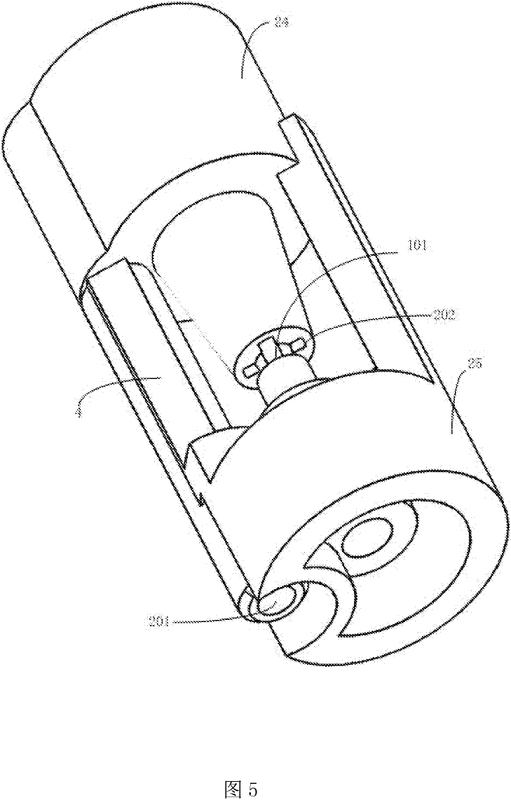

Referring to FIG. 4, as a further improvement to the above embodiment, a hollow mounting cylinder 65 is provided in the first cover 6, openings are formed in upper and lower ends of the mounting cylinder 65, the mist outlet passage 62 is formed in the mounting cylinder 65, the gas inlet passage 61 is also located in the mounting cylinder 65, and the mist outlet passage 62 is located on a periphery of the gas inlet passage 61. In some embodiments, the gas inlet passage 61 is a gas passage 10 of the atomizer, and in other embodiments, the gas inlet passage 61 may also be a gas passage communicated with the gas passage 10. The atomizer 1000 is fixed in the opening at the lower end of the mounting cylinder 65, the second valve is movably mounted in the mounting cylinder 65, the second valve includes a vent pipe 922 and a seal 923 fixed on a peripheral side of the vent pipe 922, an upper end of the vent pipe 922 penetrates through the opening at the upper end of the mounting cylinder 65 to protrude outwards, and the seal 923 is tightly fitted with an inner wall of the mounting cylinder 65, such that the seal can seal a connecting through hole 9221 in the mounting cylinder 65. In the first state, the seal 923 opens the mist outlet passage 61, and the mist formed by the atomizer 1000 can be discharged out of the atomization container through the mist outlet passage 61; in the second state, the seal 923 blocks the mist outlet passage 61, and the mist formed by the atomizer 1000 cannot be discharged out of the atomization container through the mist outlet passage 61; a communication cavity 921 is formed in the vent pipe 922, and the first valve is provided in the communication cavity 921.

As a further improvement to the above embodiment, an upper segment of the vent pipe 922 is tapered and in clearance fit with the opening at the upper end of the mounting cylinder 65, such that the vent pipe 922 can be more smoothly pressed downwards, and an opening clearance between the vent pipe 922 and a side wall of the mounting cylinder 65 can be larger; the side wall of the mounting cylinder 65 is provided with the connecting through hole 9221, the second valve can move relative to the connecting through hole 9221 under the action of external force, and in the first state, the mist outlet passage 62 is opened, and the mist formed by the atomizer 1000 can be discharged out of the atomization container through the mist outlet passage 62; in the second state, the mist outlet passage 61 is blocked, and the mist formed by the atomizer 1000 cannot be discharged out of the atomization container through the mist outlet passage 62.

As a further improvement to the above embodiment, the atomization container further includes a third cover (not shown), and the third cover is a sealing cover; that is, the atomization container may be sealed after the third cover is connected with the container body 5 or the first cover 6. The third cover covers an upper portion of the container body 5 to enclose the atomizer 1000 in the accommodating cavity, and it should be noted that the third cover may directly or indirectly cover the upper portion of the container body 5; that is, the third cover may be directly connected onto the container body 5, or other components may be provided between the container body 5 and the third cover, and the third cover is indirectly connected onto the container body 5; as long as the third cover can still enclose part or all of the atomizer in an atomizing cavity, the solution thereof falls within the protection scope of the present application. During transportation or sale, the third cover covers the upper portion of the container body 5, thus avoiding that the first valve and/or the second valve are/is opened by mistake, and thus, the liquid flows out of the container body; and when used for atomization, the atomization container is used after the third cover is removed.

Referring to FIGS. 1 to 3, as a further improvement to the above embodiment, an end of the top column 74 close to the sealing part is provided with an annular sealing groove 741, the first sealing part 91 is provided with an annular sealing portion 63, and when the second cover 7 is pressed downwards (the second cover can be pressed downwards directly or screwed to be moved downwards), the annular sealing portion 63 is hermetically connected with the annular sealing groove 741; the gas inlet passage 61 and the auxiliary gas inlet passage 71 are used for flow of the high pressure gas, the mist outlet passage 62 and the auxiliary mist outlet passage 72 are used for flow of the mist formed after atomization by the atomizer, and due to the sealed connection of the annular sealing portion 63 and the annular sealing groove 741, the gas inlet passage 61 and the auxiliary gas inlet passage 71 are isolated from the mist outlet passage 62 and the auxiliary mist outlet passage 72; that is, the gas inlet passage 61 and the auxiliary gas inlet passage 71 are communicated to form one passage, the mist outlet passage 62 and the auxiliary mist outlet passage 72 are communicated to form another passage, and the two passages are isolated to isolate the high pressure gas from the mist. Specifically, a communication port is provided in the mist outlet passage 62, and the second sealing part 92 penetrates through the communication port; in the first state, the second sealing part 92 is hermetically connected with a side wall of the communication port; in the second state, a clearance is formed between the second sealing part 92 and the side wall of the communication port, such that the mist outlet passage 62 is communicated with the auxiliary mist outlet passage 72.

As a further improvement to the above embodiment, referring to FIG. 4, the communication cavity 921 is provided in the second sealing part 92, the communication cavity 921 has one end communicated with the gas inlet passage 61 and the other end communicated with the auxiliary gas inlet passage 71, and the first sealing part 91 is located in the communication cavity 921; in the first state, the first sealing part 91 blocks the communication cavity 921; in the second state, the gas inlet passage 61, the communication cavity 921, and the auxiliary gas inlet passage 71 are communicated in order. Specifically, a notch is provided at a position of the top column 74 in contact with the first sealing part 91, the first sealing part 91 is a spherical sealing part or a conical sealing part or a hemispherical sealing part, and in the present embodiment, a lower end of the top column is set into a shape with a long side and a short side, such that a gap exists when the top column 74 is in contact with the first sealing part 91. When the first sealing part 91 and a side wall of the communication cavity 921 form sealing, the gas inlet passage 61 and the auxiliary gas inlet passage 71 are sealed; when a clearance is formed between the first sealing part 91 and the side wall of the communication cavity 921, the top column 74 and the second sealing part 92 are sealed, and the mist outlet passage is communicated with the auxiliary mist outlet passage. An end of the first sealing part 91 close to the top column 74 has a gradually reduced diameter, the second sealing part 92 seals the communication port when the first sealing part 91 and the side wall of the communication cavity 921 form sealing, and the second sealing part 92 is hermetically connected with the top column 74 when a clearance is formed between the second sealing part 92 and the communication port.

As a further improvement to the above embodiment, the container body 5 includes a bottle body portion 51 and a bottle mouth portion 52, a diameter of the bottle mouth portion 52 is less than that of the bottle body portion 51, an end of the bottle body portion 51 close to the bottle mouth portion 52 is provided with a bottle clamping groove 511, and a bottle mouth is formed on the bottle mouth portion 52; the first cover 6 is provided with an annular sealing portion 64 and a bottle clamping groove 511, the annular sealing portion 64 and the bottle mouth are fixedly snapped and connected, and the bottle clamping groove 511 and the bottle clamping groove 511 are fixedly snapped and connected. Specifically, the first cover 6 is made of an elastic plastic or plastic material and can achieve both connecting and sealing effects at the same time by means of fixed snapping and connection as well as an interference fit.

As a further improvement to the above embodiment, the mist outlet passage 62 and/or the auxiliary mist outlet passage 72 are/is curved, such that large mist droplets can fall back into the container body 5 during a mist outlet process, thus avoiding that the discharged large mist droplets fall around the container body 5 to cause pollution. The first cover 6 and the second cover 7 are provided with mutually fitted screw threads and fixedly connected through the screw threads, such that the connection is more stable. The mist outlet 73 has a mist outlet direction inclined upwards (upward inclination means that when the container is normally placed on a table, a height of a free end of the mist outlet is greater than heights of other positions of the mist outlet), and large mist droplets falling on the mist outlet 73 can flow back into the container body.

As a further improvement to the above embodiment, the atomizer is further included, the atomizer 1000 is provided in the gas inlet passage 61, the mist outlet 73 of the atomizer 1000 is communicated with the mist outlet passage 62, and the atomizer is connected with a liquid guiding part, such as a cotton core or a liquid guiding capillary tube.

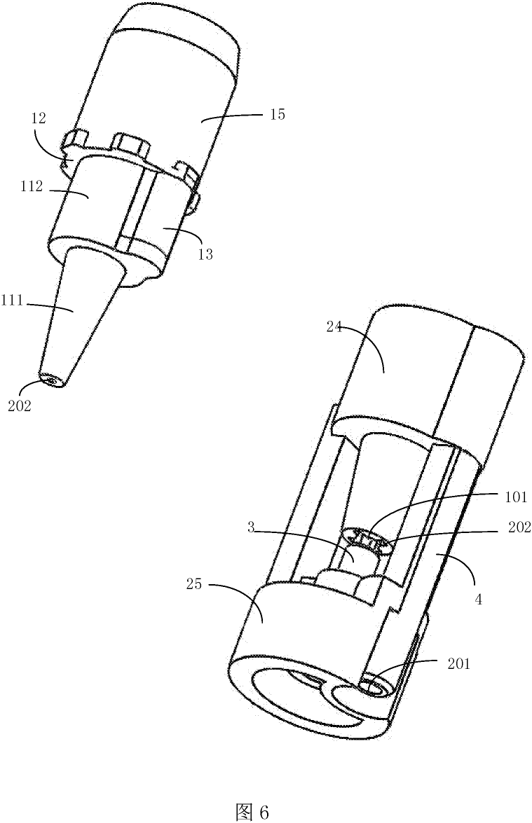

Referring to FIGS. 5 to 9, as a further improvement to the above embodiment, the atomizer 1000 includes an atomizer body 100 and a blocking part 3, the atomizer body 100 is provided with a liquid passage 20 and a gas passage 10, and in use, liquid to be atomized is introduced into the liquid passage 20, and high pressure gas is introduced into the gas passage 10. A liquid outlet 202 is provided at a tail end of the liquid passage 20, a gas outlet 101 is provided at a tail end of the gas passage 10, and the liquid outlet 202 is distributed on a periphery of the gas outlet 101; the blocking part 3 is provided opposite to the gas outlet 101 and the liquid outlet 202 and spaced apart from the gas outlet 101 and the liquid outlet 202 by a preset distance, and the blocking part 3 refracts the gas flow emitted from the gas outlet 101; that is, after the gas flow hits the blocking part, a moving direction of the gas flow is changed by the blocking part like refraction of light. The liquid outlet 202 is located downstream of the gas outlet 101 in the refraction direction; that is, the gas flow can hit the liquid outlet 202 after retracted. The high pressure gas hits the blocking part 3 after discharged from the gas passage 10, the blocking part 3 diffuses the high pressure gas all around to scatter and atomize the liquid discharged from the liquid outlet 202, the mist after the atomization is diffused all around with flow of the high pressure gas, and since the high pressure gas has a high flow velocity, a fragrance after the atomization can also be diffused all around at a high speed.

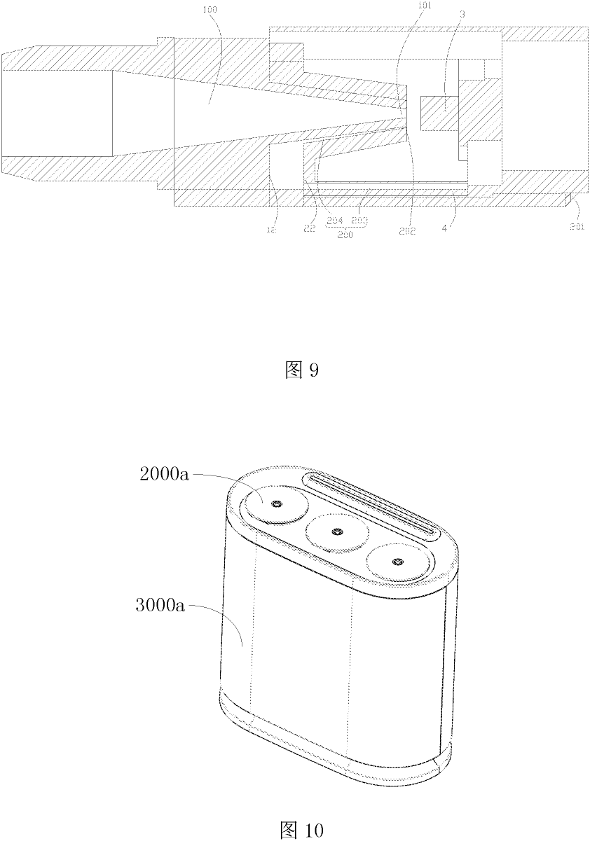

Referring to FIG. 9, as a further improvement to the above embodiment, the atomizer body 100 includes a first body 1 and a second body 2, the first body 1 and the second body 2 are detachably connected, and the first body 1 is sleeved by the second body 2; the gas passage 10 is formed in the first body 1, and the liquid passage 20 is formed in the second body 2 or an outer wall of the first body 1.

Referring to FIGS. 5 and 9, as a further improvement to the above embodiment, the liquid passage 20 includes a main flow passage 203 and branch passages 204, and plural branch passages 204 are provided and all communicated with the main flow passage 203. The plurality of branch passages 204 branch off from the main flow passage 203, and a small number of liquid inlets 201 or one liquid inlet 201 can supply liquid to the liquid outlet 202, thus reducing a number of the main flow passage 203 and facilitating processing. A liquid outlet 202 is formed in a tail end of each branch passage 204, and since the number of the main flow passages 203 is small, the main flow passages 203 can have relatively large apertures to reduce blockage of the liquid passage 20. In another embodiment, the liquid passage 20 includes a main flow passage 203 and a branch passage 204, the branch passage 204 is annular, the liquid outlet 202 is also annular, and the gas outlet is located in a middle portion of the annular liquid outlet. The liquid outlet 202 is circular, a difference between an outer diameter and an inner diameter of the annular liquid outlet 202 is 0.2-0.5 mm, preferably, 0.3 mm; the outer diameter is only a diameter of an outer edge of the circular liquid outlet, and the inner diameter is a diameter of an inner edge of the circular liquid outlet.

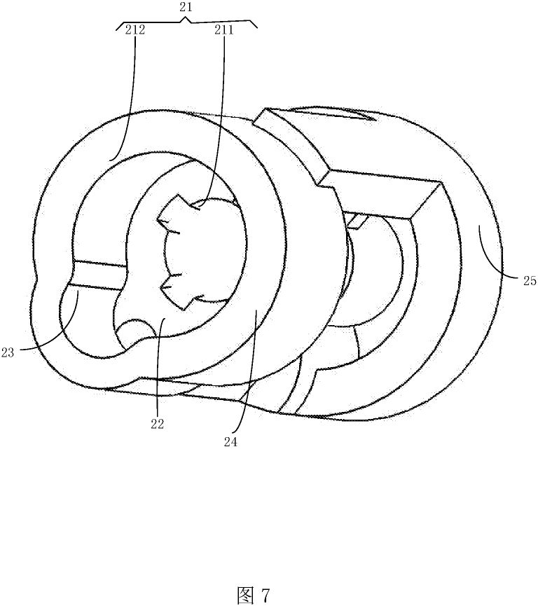

Referring to FIGS. 6 and 7, as a further improvement to the above embodiment, the second body 2 includes a main portion 24 and a cooperating portion 25, a connecting rod 4 is provided between the main portion 24 and the cooperating portion 25, the main portion 24 and the cooperating portion 25 are fixedly connected by the connecting rod 4, and a portion between the main portion 24 and the cooperating portion 25 is communicated with the outside for diffusing the mist. Specifically, the portion between the main portion 24 and the cooperating portion 25 except for the connecting rod 4 is open, thus facilitating mist diffusion. The blocking part 3 is fixed on the cooperating portion 25, and the blocking part 3 is 0.1 mm to 1 mm away from the gas outlet 101; specifically, the blocking part 3 is 0.2 mm, or 0.3 mm, or 0.4 mm, or 0.5 mm, or 0.6 mm, or 0.7 mm, or 0.8 mm, or 0.9 mm away from the gas outlet 101.

Referring to FIG. 9, as a further improvement to the above embodiment, an inlet of the main flow passage 203 is provided on the cooperating portion 25, and the main flow passage 203 extends through the connecting rod 4 to the main portion 24.

Referring to FIGS. 6 and 7, as a further improvement to the above embodiment, the second body 2 is provided with a clamping hole 21, a periphery of the clamping hole 21 is provided with a groove having the same extending direction as the clamping hole, and the groove extends to the liquid outlet 202. After the first body 1 is inserted into the clamping hole, the groove is formed as the branch passage 204. The liquid passage 20 and the gas passage 10 (especially the liquid passage 20) both have small diameters, the liquid cannot be sucked out by negative pressure if the diameter is too large, and processing is difficult if the diameter is too small. The arrangement of the liquid passage 20 and the gas passage 10 makes the formation of the liquid passage 20 and the gas passage 10 simpler, the gas outlet 101 of the gas passage 10 is small, cross sections at gas positions are quite large, and therefore, processing is convenient, and cleaning is easy. Since the branch passage 204 cannot be too large, the mode that the first body 1 and the second body 2 cooperate to form the branch passage 204 is adopted in the solution, such that the branch passage 204 is formed simply, and the branch passage 204 is easy to clean.

Referring to FIGS. 6 to 8, as a further improvement to the above embodiment, the clamping hole 21 includes a first clamping hole 211 and a second clamping hole 212 communicated with the first clamping hole 211, the second clamping hole 212 has a greater cross-sectional area than the first clamping hole 211, a first step 22 is formed between the first clamping hole 211 and the second clamping hole 212, an inlet of the branch passage 204 is located on the step, and an outlet of the main flow passage 203 is also located on the first step 22. Referring to FIG. 4, the first body 1 includes a clamping portion 11 and a connecting portion 15, and the connecting portion 15 protrudes from the clamping hole for connection with an external gas pump. The clamping portion 11 includes a first clamping portion 111 and a second clamping portion 112, the first clamping portion 111 is fitted with the first clamping hole 211, and the second clamping portion 112 is fitted with the second clamping hole 212. A second step 12 is formed between the first clamping portion 111 and the second clamping portion 112, a clearance is formed between the first step 22 and the second step 12, and the main flow passage 203 and the plurality of branch passages 204 are communicated through the clearance. The first clamping portion 111 is inserted into a first clamping groove, and the first clamping portion 111 is attached to a side wall of the first clamping groove, such that a groove located on a periphery of the first clamping groove forms the branch passage 204, and the first body 1 and the second body 2 can be fixed. The second clamping portion 112 is in interference fit with a side wall of a second clamping groove, such that the first body 1 and the second body 2 are connected more stably.

As a further improvement to the above embodiment, a third step 14 is provided between the connecting portion 15 and the clamping portion, and the third step 14 cooperates with the second step to limit a length of the clamping portion inserted into the clamping hole, such that the clearance formed by the first step 22 and the second step 12 is not too small or a case where no clearance exists between the first step 22 and the second step 12 is avoided.

As a further improvement to the above embodiment, a positioning groove 23 is provided on the second clamping hole 212, a protrusion 13 fitted with the positioning groove 23 is provided on the second clamping portion 112, and the outlet of the main flow passage 203 is located in the positioning groove 23. Relative positions of the first body 1 and the second body 2 are fixed, such that the first body 1 cannot rotate in the clamping groove of the second body 2.

As a further improvement to the above embodiment, the first clamping portion 111 is tapered, and the liquid passage 20 is also tapered, such that the groove is conveniently formed, and processing is convenient; the liquid outlet 202 of the liquid passage 20 is as close as possible to the gas outlet 101, such that an atomization effect is better. The liquid outlet 202 of the liquid passage 20 and the gas outlet 101 of the gas passage 10 are located on the same plane, and the gas flow with the flow direction changed by the blocking part 3 directly acts on the liquid discharged from the liquid outlet 202, and cannot be blocked by other objects; otherwise, the flow velocity of the gas flow is influenced, and the atomization effect is also influenced.

The gas outlet 101 is provided opposite to the blocking part 3, the liquid outlet 202 is provided on the periphery of the gas outlet 101, and the high pressure gas impacting on the blocking part 3 is diffused all around, such that the liquid discharged from the liquid outlet 202 is scattered, thereby spraying the mist all around.

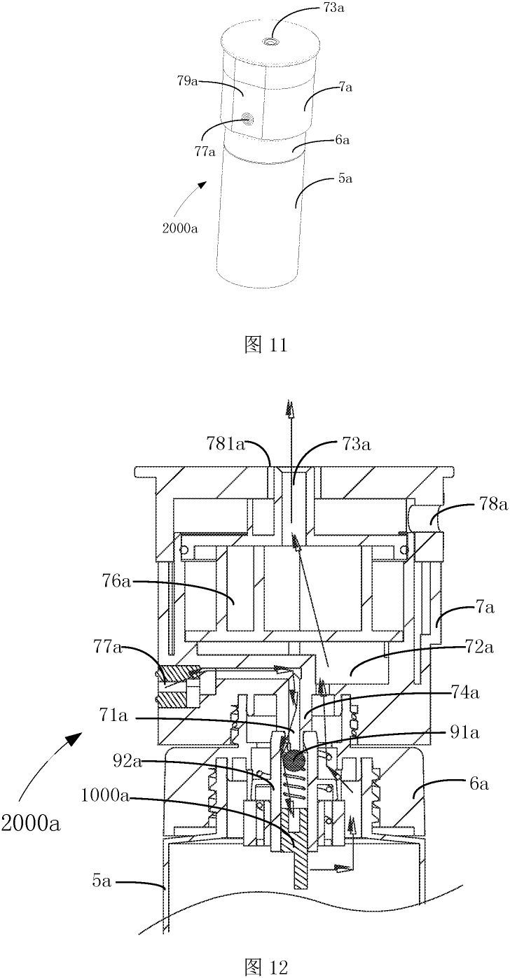

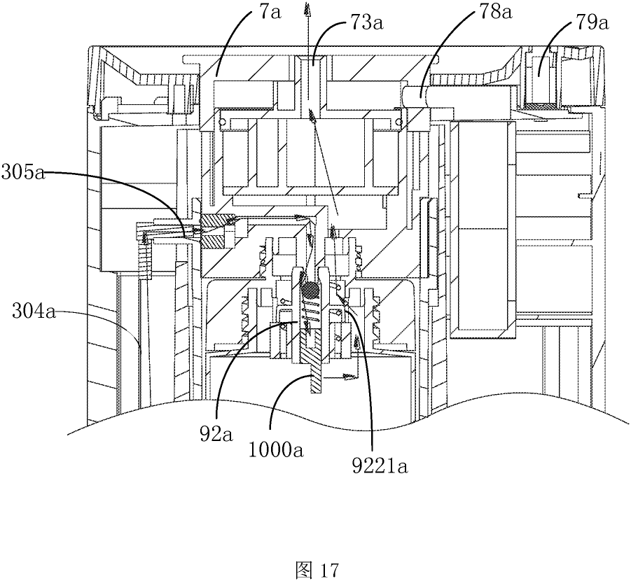

In some other embodiments, the structure of the atomization apparatus is shown in FIGS. 10 to 17. The atomization apparatus includes an atomization apparatus body 3000a and an atomization container 2000a. The atomization apparatus body 3000a is provided with a mounting hole 308a, and the mounting hole 308a is exposed outside a housing of the atomization apparatus body 3000a; that is, the mounting hole 308a is visible from the outside. The atomization container 2000a is inserted into the mounting hole 308a, and a gas pump 303a is further provided in the atomization apparatus body 3000a. The atomization container 2000a includes a container body 5a and an atomizer 1000a. The container body 5a has an accommodating cavity for containing essential oil therein. The atomization container 2000a further has a gas inlet passage 61a and a mist outlet passage 62a, and the gas inlet passage 61a has one end connected with the gas pump 303a and the other end connected with the atomizer 1000a, such that the gas pump 303a supplies a high-speed compressed gas flow to the atomizer 1000a through the gas inlet passage. The atomizer 1000a is configured to mix and atomize the gas flow provided by the gas inlet passage 61a and liquid (essential oil) in the accommodating cavity to form mist. The mist outlet passage 62a is configured to discharge the mist out of the container body 5a and finally out of the atomization apparatus body 3000a. A gas outlet connector 305a connected with the gas pump 303a is provided on a side wall of the mounting hole 308a, and the gas outlet connector 305a is connected with the gas pump 303a through a gas outlet pipe 304a. The atomization container 2000a is provided with a gas inlet connector 77a connected with the gas inlet passage 61a. When the atomization container 2000a is inserted into the mounting hole 308a, the gas inlet connector 77a is communicated with the gas outlet connector 305a. It should be noted that the connection referred to here may be indirect connection or direct connection. The atomization container 2000a has a similar structure to and a same working principle as the atomization container shown in FIGS. 1 to 9, and the working principle of the atomization container 2000a will not be repeated here in the following embodiments.

In a preferred embodiment, the atomizer 1000a is located in the accommodating cavity, and in a first state, the gas inlet passage 61a and the mist outlet passage 62 are opened, such that the gas flow of the gas pump 303a can enter the atomizer 1000a through the gas inlet passage 61a, and the mist formed by the atomizer 1000a can be discharged out of the container body 5a through the mist outlet passage 62a. In a second state, the gas inlet passage 61a and/or the mist outlet passage 62a are/is closed. In the present embodiment, in the second state, the gas inlet passage 61a and the mist outlet passage 62a are closed at the same time, and in other embodiments, one of the gas inlet passage 61a and the mist outlet passage 62a may be closed.

In a preferred embodiment, the atomization container 2000a further includes a first cover 6a, and the first cover 6a is connected with the container body 5a and closes an opening of the container body 5a. The first cover 6a and the container body 5a may be detachably connected, or formed as an integral structure by means of ultrasonic welding, or the like. The atomizer 1000a is connected with the first cover 6a, the gas inlet passage 61a and the mist outlet passage 62a are provided in the first cover 6a, and the first cover 6a is further provided with a first valve 91a and a second valve 92a. The first valve 91a is fitted with the gas inlet passage 61a and configured to open or close the gas inlet passage 61a, and the second valve 92a is fitted with the mist outlet passage 62a and configured to open or close the mist outlet passage 62a. In a free state, the first valve 91a and the second valve 92a are closed, and the first cover 6a seals the container body 5a, such that the essential oil in the container body 5a does not leak even when the container body 5a is placed upside down.

In a preferred embodiment, the first valve 91a includes a first sealing part 911a and a first elastic part 81a, the first elastic part 81a is connected with the first sealing part 911a, the second valve 92a includes a second sealing part 923a and a second elastic part 82a, and the second elastic part 82a is connected with the second sealing part 923a. When the first elastic part 81a and the second elastic part 82a are in the free state, the first sealing part 911 a closes the gas inlet passage 61a, the second sealing part 923a closes the mist outlet passage 62a, and when the first sealing part 911a and the second sealing part 923a are pressed down by external force, the gas inlet passage 61a communicates the atomizer 1000a with a space outside the container body 5a, and the mist outlet passage 62a communicates the accommodating cavity with the space outside the container body 5a.

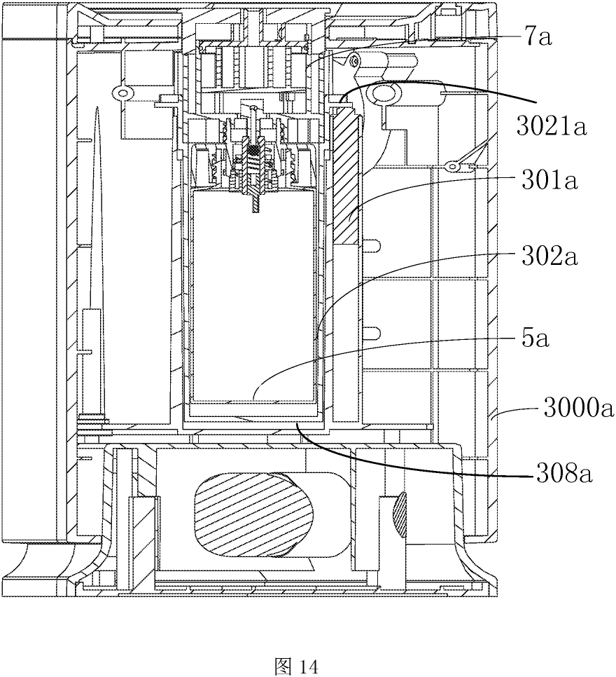

Referring to FIG. 13, in a preferred embodiment, a hollow mounting cylinder 65a is provided in the first cover 6a, openings are formed in upper and lower ends of the mounting cylinder 65a, and the second valve 92a is movably mounted in the mounting cylinder 65a. The second valve 92a includes a vent pipe 922a and a seal (i.e., the second sealing part in the above embodiment) 923a fixed to a peripheral side of the vent pipe 922a, the gas inlet passage 61a is formed in the vent pipe 922a, and the first valve 91a is provided in the gas inlet passage 61a. The mist outlet passage 62a is formed in the mounting cylinder 65a, and the mist outlet passage 62a is located outside the vent pipe 922a. An upper end of the vent pipe 922a penetrates through the opening 655a at the upper end of the mounting cylinder 65a to protrude outwards. The seal 923a is tightly fitted with an inner wall of the mounting cylinder 65a. The atomizer 1000a is fixed into the opening at the lower end of the mounting cylinder 65a and is fixed relative to the mounting cylinder 65a. In the present embodiment, the atomizer 1000a is fixed to a lower end of the vent pipe 922a, and is movable up and down with the vent pipe 922a relative to the mounting cylinder 65a and the container body 5a. In the first state, the seal 923a opens the mist outlet passage 62a, and the mist formed by the atomizer 1000a can be discharged out of the atomization container 2000a through the mist outlet passage 62a. In the second state, the seal 923a blocks the mist outlet passage 62a, and the mist formed by the atomizer 1000a cannot be discharged out of the atomization container 2000a through the mist outlet passage 62a.

In a preferred embodiment, an upper segment of the vent pipe 922a is in clearance fit with the opening 655a at the upper end of the mounting cylinder 65a, a connecting through hole 9221a is formed in a side wall of the mounting cylinder 65a, the connecting through hole 9221a is communicated with the accommodating cavity, the second valve 92a can move relative to the connecting through hole 9221a under the action of external force, and in the first state, the seal 923a opens the mist outlet passage 62a between the connecting through hole 9221a and the opening 655a at the upper end of the mounting cylinder 65a, and the mist formed by the atomizer 1000a enters the mounting cylinder 65a from the accommodating cavity through the connecting through hole 9221 a, and is discharged out of the first cover 6a through the opening at the upper end of the mounting cylinder 65a; in the second state, the seal 923a closes the mist outlet passage 62a between the connecting through hole 9221a and the opening 655a at the upper end of the mounting cylinder 65a, and the mist formed by the atomizer 1000a is sealed in the accommodating cavity and may partially enter the mounting cylinder 65a.

In a preferred embodiment, the atomization container 2000a further includes a third cover (not shown), the third cover is a sealing cover, the third cover is detachably connected with the first cover 6a, and when the third cover is hermetically connected with the first cover 6a, the atomizer 1000a, the gas inlet passage 61a and the mist outlet passage 62a are sealed in the accommodating cavity. During transportation or sale of the atomization container 2000a, the third cover is connected to an upper portion of the first cover 6a, thus avoiding that the first valve 91a and/or the second valve 92a are/is opened by mistake, and thus, the liquid flows out of the container body 5a; when used for atomization, the atomization container 2000a is used after the third cover is removed.

Referring to FIG. 12, in a preferred embodiment, the atomization container 2000a further includes a second cover 7a, and the second cover 7a is provided with a top column 74a, an auxiliary gas inlet passage 71a, an auxiliary mist outlet passage 72a and a mist outlet 73a. The auxiliary gas inlet passage 71a is provided in the top column 74a, and the mist outlet 73a is communicated with the auxiliary mist outlet passage 72a. When the second cover 7a is connected with the first cover 6a, the top column 74a presses down the first valve 91a and the second valve 92a, such that the auxiliary gas inlet passage 71a is communicated with the gas inlet passage 61a, the auxiliary mist outlet passage 72a is communicated with the mist outlet passage 62a, and the gas inlet passage 61a and the auxiliary gas inlet passage 71a are isolated from the mist outlet passage 62a and the auxiliary mist outlet passage 72a. The second cover 7a and the first cover 6a may be connected by screw threads. In a further preferred embodiment, a filter 76a may be further provided in the second cover 7a, and the filter 76a is configured to filter larger mist particles. The filter 76a may have a labyrinth structure.

In a preferred embodiment, the first cover 6a is provided therein with a hollow mounting cylinder 65a, openings are formed in upper and lower ends of the mounting cylinder 65a, the second valve 92a is vertically movably mounted in the mounting cylinder 65a, the second valve 92a includes a vent pipe 922a and a seal 923a fixed to a peripheral side of the vent pipe 922a, the gas inlet passage 61a is formed in the vent pipe 922a, the first valve 91a is provided in the gas inlet passage 61a, the mist outlet passage 62a is formed in the mounting cylinder 65a, and the mist outlet passage 62a is located outside the vent pipe 922a. An upper end of the vent pipe 922a penetrates through the opening 655a at the upper end of the mounting cylinder 65a to protrude outwards, the seal 923a is tightly fitted with an inner wall of the mounting cylinder 65a, and the atomizer 1000a is fixed in the opening at the lower end of the mounting cylinder 65a or fixed at a lower end of the vent pipe 922a. When the second cover 7a is connected with the first cover 6a, the top column 74a presses the second valve 92a downwards to open the mist outlet passage 62a, and a lower end portion of the top column 74a is inserted into the gas inlet passage 61a of the second valve 92a and abuts against the first valve 91a, such that the auxiliary gas inlet passage 71a and the gas inlet passage 61a form a butt joint and are communicated.

In a preferred embodiment, the first valve 91a includes a first sealing part 911a and a first elastic part 81a, the first elastic part 81a is connected with the first sealing part 911a, the first sealing part 911a is a spherical sealing part, the first sealing part 911a is pressed down when the lower end portion of the top column 74a is inserted into the second valve 92a, a notch is formed at a position of the lower end portion of the top column 74a in contact with the first sealing part, and the gas inlet passage 61a is communicated with the auxiliary gas inlet passage 71a through the notch.

In a preferred embodiment, the atomizer 1000a has a gas passage and a liquid passage, the gas passage has a gas inlet and a gas outlet which are communicated with each other, the liquid passage has a liquid inlet and a liquid outlet which are communicated with each other, the gas outlet is adjacent to the liquid outlet, the atomizer 1000a further includes a pipette, the pipette has one end extending to a bottom of the accommodating cavity and the other end communicated with the liquid inlet, the gas inlet is connected with the gas inlet passage 61a, the gas outlet is located above a liquid level of the accommodating cavity, and the gas flow provided through the gas inlet passage 61a and the liquid in the accommodating cavity are mixed and atomized near the gas outlet and discharged into the accommodating cavity. In other embodiments, the atomizer 1000a may have other structures.

In a preferred embodiment, a gas inlet connector 77a is provided on the second cover 7a, the gas inlet connector 77a is connected with the auxiliary gas inlet passage 71a, and the second cover 7a is provided with an auxiliary air outlet 781a surrounding the mist outlet 73a and an auxiliary air inlet 78a located on one side of the second cover 7a. The auxiliary air inlet 78a is communicated with the auxiliary air outlet 781a, the atomization apparatus body 3000a is further provided with an auxiliary air blowing apparatus 79a, the auxiliary air blowing apparatus 79a is opposite to the auxiliary air inlet 78a, and air is blown into the auxiliary air inlet 78a and out of the atomization apparatus body 3000a through the auxiliary air outlet 781a. The auxiliary air blowing apparatus 79a may be a fan for forming auxiliary air flow near the mist outlet 73a, such that the essential oil mist can travel farther.

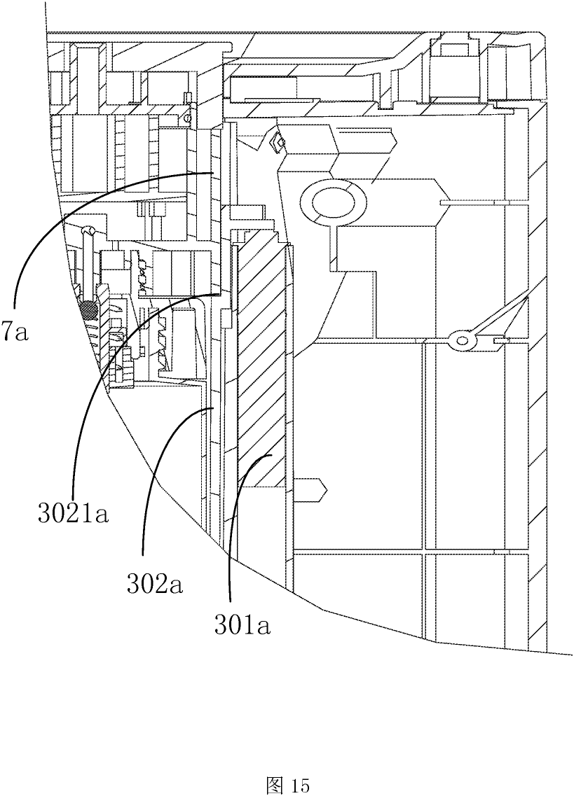

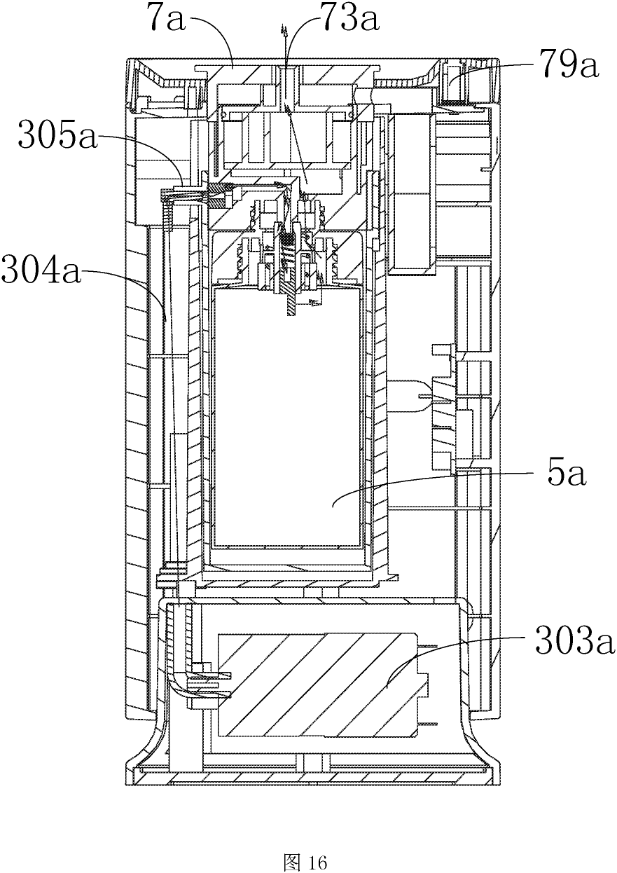

Referring to FIGS. 14 and 15, in a preferred embodiment, the atomization apparatus body 3000a includes an inner cylinder 302a and a rebounder 301 a, and the inner cylinder 302a is inserted into the mounting hole 308a. The inner cylinder 302a is provided with a rebounding portion 3021a protruding towards a periphery of the inner cylinder 302a, and the rebounder 301a is mounted in the atomization apparatus body 3000a, is located outside the inner cylinder 302a, and abuts against the rebounding portion 3021a from bottom to top. The atomization container 2000a is partially inserted into the inner cylinder 302a. The rebounder 301a has a compressed state and a bounced state under the action of external force, and alternately gets into the compressed state and the bounced state under the action of the external force; that is, the rebounder is pressed once to be in the compressed state and then pressed once again to be in the bounced state, thus driving the inner cylinder 302a and the atomization container 2000a to move up and down in the mounting hole 308a. A height of the atomization container 2000a relative to the mounting hole 308a in the bounced state of the rebounder 301a is greater than a height of the atomization container 2000a relative to the mounting hole 308a in the compressed state of the rebounder 301a, thereby facilitating removal of the atomization container 2000a from the mounting hole 308a.

In a further preferred embodiment, the atomization container 2000a further includes a first cover 6a, the first cover 6a is connected with the container body 5a and closes an opening of the container body 5a, the atomizer 1000a is connected with the first cover 6a, the gas inlet passage 61a and the mist outlet passage 62a are provided in the first cover 6a, the first cover 6a is further provided with a first valve 91a and a second valve 92a, the first valve 91a is fitted with the gas inlet passage 61a and configured to open or close the gas inlet passage 61a, and the second valve 92a is fitted with the mist outlet passage 62a and configured to open or close the mist outlet passage 62a;
the atomization container 2000a further includes a second cover 7a, the second cover 7a has a greater outer diameter than the first cover 6a, the second cover 7a is provided with a top column 74a, an auxiliary gas inlet passage 71a, an auxiliary mist outlet passage 72a and a mist outlet 73a, the auxiliary gas inlet passage 71a is provided in the top column 74a, the mist outlet 73a is communicated with the auxiliary mist outlet passage 72a, the second cover 7a is connected with the first cover 6a, and when the top column 74a presses the first valve 91a and the second valve 92a downwards, the auxiliary gas inlet passage 71a is communicated with the gas inlet passage 61a, the auxiliary mist outlet passage 72a is communicated with the mist outlet passage 62a, and the gas inlet passage 61a and the auxiliary gas inlet passage 71a are isolated from the mist outlet passage 62a and the auxiliary mist outlet passage 72a;
the first cover 6a is inserted into the inner cylinder 302a, the second cover 7a is located outside the inner cylinder 302a, a supporting step 3021a is provided on an inner wall of the inner cylinder 302a, and the second cover 7a is supported on the supporting step 3021a. By providing the supporting step 3021a, the atomization container 2000a is more easily positioned.

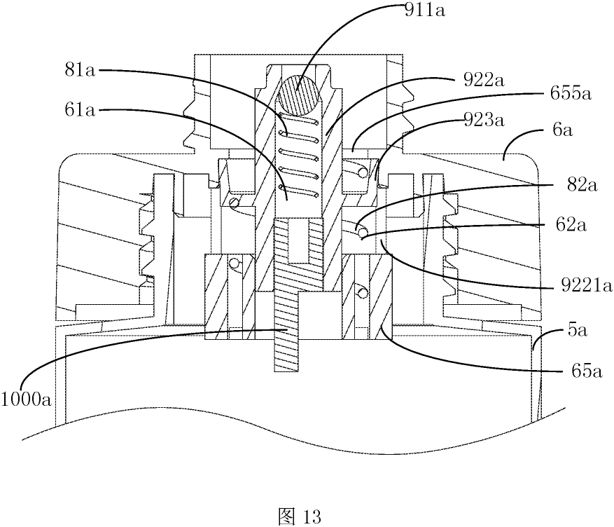

In a preferred embodiment, the atomization container 2000a further includes a first cover 6a, the first cover 6a is connected with the container body 5a and closes an opening of the container body 5a, the atomizer 1000a is connected with the first cover 6a, the gas inlet passage 61a and the mist outlet passage 62a are provided in the first cover 6a, the first cover 6a is further provided with a first valve 91a and a second valve 92a, the first valve 91a is fitted with the gas inlet passage 61a and configured to open or close the gas inlet passage 61a, and the second valve 92a is fitted with the mist outlet passage 62a and configured to open or close the mist outlet passage 62a;
the atomization container 2000a further includes a second cover 7a, the second cover 7a is provided with a top column 74a, an auxiliary gas inlet passage 71a, an auxiliary mist outlet passage 72a and a mist outlet 73a, the auxiliary gas inlet passage 71a is provided in the top column 74a, the mist outlet 73a is communicated with the auxiliary mist outlet passage 72a, the second cover 7a is connected with the first cover 6a, and when the top column 74a presses the first valve 91a and the second valve 92a downwards, the auxiliary gas inlet passage 71a is communicated with the gas inlet passage 61a, the auxiliary mist outlet passage 72a is communicated with the mist outlet passage 62a, and the gas inlet passage 61a and the auxiliary gas inlet passage 71a are isolated from the mist outlet passage 62a and the auxiliary mist outlet passage 72a;
referring to FIG. 11, the second cover 7a is columnar, an outer side surface of the second cover 7a fitted with the mounting hole 308a includes a first arc surface region and a first plane region 79a connected with the first arc surface region, an inner wall of the mounting hole 308a is correspondingly provided with a second arc surface region and a second plane region connected with the second arc surface region, and the first arc surface region and the first plane region 79a are in correspondingly fitting contact with the second arc surface region and the second plane region respectively. The gas inlet connector 77a and the gas outlet connector 305a are provided in the first plane region 79a and the second plane region respectively. Since the inner wall of the mounting hole 308a and the outer side surface of the second cover 7a are both in the form of a curved surface plus a plane, the second cover 7a can be inserted into the mounting hole 308a only when the first plane region 79a is aligned with the second plane region, which can ensure that the gas inlet connector 77a can be aligned with the gas outlet connector 305a.

In a preferred embodiment, the atomization apparatus includes a plurality of atomization containers 2000a, a plurality of mounting holes 308a are correspondingly formed in the atomization apparatus body 3000a, and each atomization container 2000a is connected with a corresponding gas pump 303a, or the plurality of atomization containers 2000a are connected with the same gas pump 303a; the atomization apparatus further includes auxiliary air blowing apparatuses 79a, each atomization container 2000a is provided with a mist outlet 73a and an auxiliary air outlet 781a surrounding the mist outlet 73a, the mist outlet 73a is communicated with the mist outlet passage 62a, the auxiliary air outlet 781a of each atomization container 2000a is connected with one corresponding auxiliary air blowing apparatus 79a, or the auxiliary air outlets 781a of the plural atomization containers 2000a are connected with the same auxiliary air blowing apparatus 79a. That is, the atomization containers 2000a and the gas pumps 303a may be in a one-to-one correspondence relationship, or a plurality of atomization containers 2000a may be connected with one gas pump 303a, as is the correspondence relationship between the atomization containers 2000a and the auxiliary air blowing apparatuses 79a.

The atomization container according to the present application is internally provided with the atomizer, and therefore more easily mounted in the atomization apparatus; moreover, the atomizer is replaced while the atomization container is replaced, such that the atomizer of the atomization apparatus is always in a good working state, and an atomization effect is better.

The above is only preferred embodiments of the present application and thus not intended to limit the scope of the present application, and equivalent structure or equivalent flow transformations made by virtue of the description and drawings of the present application, or direct or indirect applications to other related arts shall similarly fall within the scope of protection of the present application.

What is claimed is:

1. An atomization container, comprising:
a container body and an atomizer where the container body has an accommodating cavity therein the atomizer comprised in the accommodating cavity,
a gas inlet passage and a mist outlet passage where the gas inlet passage has one end configured to be connected with an external gas source and another other end connected with the atomizer to provide gas flow for the atomizer;
wherein the atomizer is configured to mix and atomize the gas flow provided through the gas inlet passage and a liquid in the accommodating cavity to form a mist, and the mist outlet passage is configured to discharge the mist out of the container body;
wherein in a first state, the gas inlet passage and the mist outlet passage are opened so that gas flow of a gas pump can enter the atomizer through the gas inlet passage and the mist formed by the atomizer can be discharged out of the container body through the mist outlet passage; and
wherein in a second state, one of the gas inlet passage, the mist outlet passage, or both the gas inlet passage and the mist outlet passage are closed.

2. The atomization container of claim 1, wherein the atomization container further comprises:
a first cover where the first cover is connected with the container body and closes an opening of the container body, the atomizer connected with the first cover and where the gas inlet passage and the mist outlet passage are provided in the first cover;
wherein the first cover is further provided with a first valve and a second valve, the first valve fitted with the gas inlet passage and configured to open or close the gas inlet passage, and the second valve fitted with the mist outlet passage and configured to open or close the mist outlet passage; and
wherein, in a free state, the first valve and the second valve are closed, the first cover seals the container body, the second valve is movably mounted in the first cover, and the atomizer is fixed on the second valve and can move with the second valve relative to the container body.

3. The atomization container of claim 2, further comprising:
wherein the first valve comprises a first sealing part and a first elastic part where the first elastic part is connected with the first sealing part;
wherein the second valve comprises a second sealing part and a second elastic part, the second elastic part connected with the second sealing part;
wherein when the first elastic part and the second elastic part are in a free state, the first sealing part closes the gas inlet passage, and the second sealing part closes the mist outlet passage; and
wherein when the first sealing part and the second sealing part are pressed down by an external force, the gas inlet passage communicates the atomizer with a space outside the container body, and the mist outlet passage communicates the accommodating cavity with the space outside the container body.

4. The atomization container of claim 2, further comprising:
wherein a hollow mounting cylinder is provided in the first cover;
openings are formed in an upper end and a lower end of the mounting cylinder;
the second valve is movably mounted in the mounting cylinder;
the second valve comprises a vent pipe and a seal fixed on a peripheral side of the vent pipe;
the gas inlet passage is formed in the vent pipe;
the first valve is provided in the gas inlet passage;
the mist outlet passage is formed in the mounting cylinder, and the mist outlet passage is located outside the vent pipe;
wherein an upper end of the vent pipe penetrates through an opening at the upper end of the mounting cylinder to protrude outwards;
the seal is tightly fitted with an inner wall of the mounting cylinder;
the atomizer is fixed at a lower end of the vent pipe;
in a first state, the seal opens the mist outlet passage and the mist formed by the atomizer can be discharged out of the atomization container through the mist outlet passage; and
in a second state, the seal blocks the mist outlet passage and the mist formed by the atomizer cannot be discharged out of the atomization container through the mist outlet passage.

5. The atomization container of claim 4, further comprising:
wherein an upper segment of the vent pipe is in clearance fit with the opening at the upper end of the mounting cylinder;
a connecting through hole is formed in a side wall of the mounting cylinder, the connecting through hole communicating with the accommodating cavity;
the second valve can move relative to the connecting through hole under an action of an external force;
in a first state, the seal opens a passage from the connecting through hole to the opening at the upper end of the mounting cylinder, the mist formed by the atomizer enters the mounting cylinder from the accommodating cavity through the connecting through hole and is discharged out of the first cover through the opening at the upper end of the mounting cylinder; and
in a second state, the seal closes the passage from the connecting through hole to the opening at the upper end of the mounting cylinder and the mist formed by the atomizer is sealed in the accommodating cavity.

6. The atomization container of claim 2, further comprising:
wherein the atomization container further comprises a third cover where the third cover is a sealing cover and detachably connected with the first cover; and
when the third cover is hermetically connected with the first cover, the atomizer, the gas inlet passage, and the mist outlet passage are sealed in the accommodating cavity.

7. The atomization container of claim 2, further comprising:
wherein the atomization container further comprises a second cover where the second cover is provided with a top column, an auxiliary gas inlet passage, an auxiliary mist outlet passage and a mist outlet;
wherein the auxiliary gas inlet passage is provided in the top column, the mist outlet is communicated with the auxiliary mist outlet passage; and
when the second cover is connected with the first cover, the top column presses down the first valve and the second valve such that the auxiliary gas inlet passage is in communication with the gas inlet passage, the auxiliary mist outlet passage is in communication with the mist outlet passage, and the gas inlet passage and the auxiliary gas inlet passage are isolated from the mist outlet passage and the auxiliary mist outlet passage.

8. The atomization container of claim 7, further comprising:
wherein a hollow mounting cylinder is provided in the first cover;
openings are formed in an upper end and a lower end of the mounting cylinder;
the second valve is vertically movably mounted in the mounting cylinder;
the second valve comprises a vent pipe and a seal fixed on a peripheral side of the vent pipe;
the gas inlet passage is formed in the vent pipe;
the first valve is provided in the gas inlet passage;
the mist outlet passage is formed in the mounting cylinder;
the mist outlet passage is located outside the vent pipe;
an upper end of the vent pipe penetrates through an opening at the upper end of the mounting cylinder to protrude outwards;
the seal is tightly fitted with an inner wall of the mounting cylinder;
the atomizer is fixed at a lower end of the vent pipe; and
when the second cover is connected with the first cover, the top column presses down the second valve to open the mist outlet passage and a lower end portion of the top column is inserted into the second valve and abuts against the first valve such that the auxiliary gas inlet passage and the gas inlet passage form a butt joint and are in communication.

9. The atomization container of claim 8, further comprising:
wherein the first valve comprises a first sealing part and a first elastic part where the first elastic part is connected with the first sealing part and where the first sealing part is a spherical sealing part; and
when the lower end portion of the top column is inserted into the second valve, the first sealing part is pressed downwards, a notch is formed at a position of the lower end portion of the top column in contact with the first sealing part, and the gas inlet passage is in communication with the auxiliary gas inlet passage through the notch.

10. The atomization container of claim 7, further comprising:
wherein the second cover is provided with a gas inlet connector;
the gas inlet connector is connected with the auxiliary gas inlet passage;
the second cover is provided with an auxiliary air outlet surrounding the mist outlet and an auxiliary air inlet located on one side of the second cover; and
the auxiliary air inlet is in communication with the auxiliary air outlet.

11. The atomization container of claim 1, further comprising:
wherein the atomizer has a gas passage and a liquid passage where the gas passage has a gas inlet and a gas outlet which are in communication with each other and the liquid passage has a liquid inlet and a liquid outlet which are in communication with each other;
the gas outlet is adjacent to the liquid outlet;
the atomizer further comprising a pipette, the pipette has having one end extending to a bottom of the accommodating cavity and another end in communication with the liquid inlet;
the gas inlet is connected with the gas inlet passage;
the gas outlet is located above a liquid level of the accommodating cavity; and
a gas flow provided by the gas inlet passage and a liquid in the accommodating cavity are is mixed and atomized near the gas outlet and discharged into the accommodating cavity.

12. An atomization apparatus, comprising:
an atomization apparatus body and an atomization container where the atomization apparatus body is provided with a mounting hole, the atomization container is inserted into the mounting hole, and a gas pump is further provided in the atomization apparatus body; and
wherein the atomization container is the atomization container of claim 1, where the gas inlet passage has one end connected with the gas pump and the other end connected with the atomizer such that the gas pump provides gas flow for the atomizer through the gas inlet passage and the mist is discharged out of the container body through the mist outlet passage and finally discharged out of the atomization apparatus body.

13. The atomization apparatus of claim 12, further comprising:
wherein the atomizer has a gas passage and a liquid passage where the gas passage has a gas inlet and a gas outlet which are in communication with each other and the liquid passage has a liquid inlet and a liquid outlet which are in communication with each other;
the gas outlet is adjacent to the liquid outlet;
the atomizer further comprises a pipette having one end extending to a bottom of the accommodating cavity and another end in communication with the liquid inlet;
the gas inletconnected with the gas inlet passage;
the gas outlet is located above a liquid level of the accommodating cavity, and a gas flow provided by the gas inlet passage and a liquid in the accommodating cavity are mixed and atomized near the gas outlet and discharged into the accommodating cavity;
the atomizer comprises an atomizer body and a blocking part where and the liquid passage and the gas passage are provided on the atomizer body;
the liquid outlet is provided at a tail end of the liquid passage;
the gas outlet is provided at a tail end of the gas passage;
the liquid outlet is distributed on a periphery of the gas outlet;
the blocking part is provided opposite to the gas outlet and the liquid outlet and spaced apart from the gas outlet and the liquid outlet by a preset distance where the blocking part refracts a gas flow emitted from the gas outlet, and the liquid outlet is located downstream of the gas outlet in a refracting direction;
the atomization apparatus body comprises a first body and a second body where the first body and the second body are detachably connected and the first body is sleeved by the second body; and
the gas passage is formed in the first body and the liquid passage is formed in the second body or in an outer wall of the first body.

14. The atomization apparatus of claim 12, wherein a gas outlet connector connected with the gas pump is provided on a side wall of the mounting hole, the atomization container is provided with a gas inlet connector connected with the gas inlet passage, and when the atomization container is inserted into the mounting hole, the gas inlet connector is communicated with the gas outlet connector.

15. The atomization apparatus of claim 14, wherein the atomization container further comprises:
a first cover connected with the container body and closing an opening of the container body;
the atomizer connected with the first cover;
the gas inlet passage and the mist outlet passage provided in the first cover;
the first cover further comprising a first valve and a second valve, the first valve fitted with the gas inlet passage and configured to open or close the gas inlet passage, and the second valve fitted with the mist outlet passage and configured to open or close the mist outlet passage;
in a free state, the first valve and the second valve are closed and the first cover seals the container body;
the atomization container further comprising:
a second cover comprising a top column, an auxiliary gas inlet passage, an auxiliary mist outlet passage and a mist outlet
wherein the auxiliary gas inlet passage is provided in the top column and the mist outlet is communicated with the auxiliary mist outlet passage;
when the second cover is connected with the first cover, the top column presses down the first valve and the second valve such that the auxiliary gas inlet passage is in communication with the gas inlet passage, the auxiliary mist outlet passage is in communication with the mist outlet passage, and the gas inlet passage and the auxiliary gas inlet passage are isolated from the mist outlet passage and the auxiliary mist outlet passage;
the gas inlet connector is provided on the second cover and connected with the auxiliary gas inlet passage;
the second cover is provided with an auxiliary air outlet surrounding the mist outlet and an auxiliary air inlet located on one side of the second cover where the auxiliary air inlet is in communication with the auxiliary air outlet; and
wherein the atomization apparatus body is further provided with an auxiliary air blowing apparatus opposite to the auxiliary air inlet and configured to blow air into the auxiliary air inlet and out of the atomization apparatus body through the auxiliary air outlet.

16. The atomization apparatus of claim 12, further comprising:
wherein the atomization apparatus body comprises an inner cylinder and a rebounder;
the inner cylinder is inserted into the mounting hole;
the inner cylinder is provided with a rebounding portion protruding towards a periphery of the inner cylinder;
the rebounder is mounted in the atomization apparatus body, is located outside the inner cylinder, and abuts against the rebounding portion from bottom to top;
the atomization container is partially inserted into the inner cylinder;
the rebounder has a compressed state and a bounced state under an action of an external force; and
a height of the atomization container relative to the mounting hole in the bounced state of the rebounder is higher than a height of the atomization container relative to the mounting hole in the compressed state of the rebounder.

17. The atomization apparatus of claim 16, further comprising:
wherein the atomization container further comprises a first cover connected with the container body and closing an opening of the container body;
the atomizer is connected with the first cover;
the gas inlet passage and the mist outlet passage are provided in the first cover;
the first cover is further provided with a first valve and a second valve where the first valve is fitted with the gas inlet passage and configured to open or close the gas inlet passage, and the second valve is fitted with the mist outlet passage and configured to open or close the mist outlet passage;
the atomization container further comprising:
a second cover with a greater outer diameter than the first cover;
the second cover comprising a top column, an auxiliary gas inlet passage, an auxiliary mist outlet passage and a mist outlet;
the auxiliary gas inlet passage is provided in the top column U;
the mist outlet is in communication with the auxiliary mist outlet passage;
the second cover is connected with the first cover;
the top column presses down the first valve and the second valve, such that the auxiliary gas inlet passage is in communication with the gas inlet passage, the auxiliary mist outlet passage is in communication with the mist outlet passage, and the gas inlet passage and the auxiliary gas inlet passage are isolated from the mist outlet passage and the auxiliary mist outlet passage;
the first cover is inserted into the inner cylinder;
the second cover is located outside the inner cylinder;
a supporting step is provided on an inner wall of the inner cylinder; and
the second cover is supported on the supporting step.

18. The atomization apparatus of claim 12, wherein the atomization container further comprises:
a first cover connected with the container body and closing an opening of the container body;
the atomizer is connected with the first cover;
the gas inlet passage and the mist outlet passage are provided in the first cover;
the first cover further comprising a first valve and a second valve, the first valve is fitted with the gas inlet passage and configured to open or close the gas inlet passage, and the second valve fitted with the mist outlet passage and configured to open or close the mist outlet passage;
the atomization container further comprising a second cover comprising a top column, an auxiliary gas inlet passage, an auxiliary mist outlet passage and a mist outlet;
the auxiliary gas inlet passage is provided in the top column;
the mist outlet is in communication with the auxiliary mist outlet passage;
the second cover connected with the first cover;
and-the top column presses down the first valve and the second valve such that the auxiliary gas inlet passage is in communication with the gas inlet passage, the auxiliary mist outlet passage is in communication with the mist outlet passage, and the gas inlet passage and the auxiliary gas inlet passage are isolated from the mist outlet passage and the auxiliary mist outlet passage;
the second cover is columnar;
an outer side surface of the second cover fitted with the mounting hole comprises a first arc surface region and a first plane region connected with the first arc surface region;
an inner wall of the mounting hole is correspondingly provided with a second arc surface region and a second plane region connected with the second arc surface region wherein the first arc surface region and the first plane region are in corresponding fitting contact with the second arc surface region and the second plane region, respectively; and
a gas inlet connector and a gas outlet connector are provided on the first plane region and the second plane region, respectively.

19. The atomization apparatus of claim 12, further comprising:
wherein the atomization apparatus comprises a plurality of atomization containers, a plurality of mounting holes are correspondingly formed in the atomization apparatus body, and each atomization container is connected with a corresponding gas pump or the plurality of atomization containers are connected with the same gas pump;
the atomization apparatus further comprising at least one auxiliary air blowing apparatus;
each atomization container provided with a mist outlet and an auxiliary air outlet surrounding the mist outlet where the mist outlet is in communication with the mist outlet passage; and
the auxiliary air outlet of each atomization container is connected with one corresponding auxiliary air blowing apparatus or auxiliary air outlets of the plurality of atomization containers are connected with the same auxiliary air blowing apparatus.
